(19) United States
(12) Patent Application Publication
(10) Pub. No.: US 2006/0063998 A1
(43) Pub. Date: Mar. 23, 2006

(54) Navigation and visualization of an access needle system

(76) Inventors: Ron von Jako, Melrose, MA (US); David Keith Corbett, Lawrence, MA (US); Lewis J. Levine, Weston, MA (US); Ella Zaslavsky, Marblehead, MA (US)

Correspondence Address: MCANDREWS HELD & MALLOY, LTD, 500 WEST MADISON STREET, SUITE 3400, CHICAGO, IL, 60661, US

(21) Appl. No.: 10/945,699

(22) Filed: Sep. 21, 2004

(51) Int. Cl. A61B 5/05

(52) U.S. Cl. 600/407

(57) ABSTRACT

A system for navigating and guiding a surgical instrument during a surgical procedure. A transmitter is positioned near a patient's anatomy. A receiver is attached to the surgical instrument. The position of the surgical instrument is tracked using the transmitter and receiver. A processing unit determines the location of the surgical instrument relative to the patient's anatomy and produces images with a pictorial representation of the surgical instrument and the patient's anatomy. The images are displayed on a display. A surgeon can view the images on the display and guide the surgical instrument into position inside the patient. The surgical instrument can be guided inside a patient without continuously producing real time x-ray exposures. Positions of the surgical instrument can be saved and displayed on the display along with the pictorial representation of the surgical instrument in its current position.

### BACKGROUND OF THE INVENTION

The present invention generally relates to navigation and visualization of a needle and stylet system. In particular, the invention relates to a method and system for electromagnetically guiding a needle and stylet to a region of interest in a patient's anatomy.

A needle and stylet combination may be used for performing various orthopedic and interventional procedures. For example, a needle and stylet may be used for procedures such as bone biopsies, guide wire placement, injections, and aspirations. Typically, the needle and stylet are guided to a region of interest in a patient's anatomy by either conducting open surgery and visually guiding the needle and stylet with line-of-sight techniques or by observing the manipulation of the needle and stylet in continuous real time x-ray images during the course of a procedure.

While a needle and stylet can be guided by line-of-sight techniques during open surgery, there are drawbacks to open surgery. First, open surgery requires larger incisions and increases patient recovery time following a procedure. Second, a surgeon may be forced to maneuver around various obstructions to maintain an unobstructed view. Maneuvering around obstructions and changing positions can become cumbersome if a surgeon has to guide the needle and stylet to various locations along the patient's anatomy. The difficulty of which can be multiplied two-fold if a surgeon desires to return the needle and stylet to a prior position and orientation.

Some of the drawbacks to open surgery can be solved using continuous real time x-ray imaging to observe the movement of a needle and stylet during a procedure. To begin the procedure, a small incision is made in the patient's skin and the needle and stylet are inserted in the incision. The patient is already situated on an x-ray table and real time x-ray images are taken of the patient as the procedure is performed. A C-arm imaging device can be used to provide views of the patient's anatomy and the needle and stylet from various angles. By observing the movement of the needle and stylet on a display showing the real time x-rays of the patient's anatomy and the needle/stylet combination, a surgeon can guide the needle and stylet to a region of interest. However, by using continuous real time images to guide the needle and stylet, the surgeon's hands and the patient are exposed to x-rays throughout the course of the procedure. For surgeons that have to routinely perform such procedures, the x-ray doses can become excessive and unhealthy. Also, updating the x-ray images may require rotation of the C-arm during a procedure and require medical personnel to relocate or reposition during the imaging sequence risk contaminating the sterile field.

Consequently, a need exists for a method and system for navigating and guiding a needle and stylet that is minimally invasive and minimizes x-ray exposure of patients and medical personnel. Additionally, a need exists for a method and system for navigating and guiding a needle and stylet that reduces dependence upon line-of-sight techniques or unobstructed paths.

### BRIEF SUMMARY OF THE INVENTION

A preferred embodiment of the present invention provides a system for navigating and guiding a medical instrument during a medical procedure. A transmitter is positioned near a patient's anatomy. A receiver is attached to the medical instrument. The position of the medical instrument is tracked using the transmitter and receiver. A processing unit determines the location of the medical instrument relative to the patient's anatomy and produces display images with a pictorial representation of the medical instrument and the patient's anatomy. The images are displayed on a monitor display.

A surgeon and other medical personnel can view the images on the display and guide surgical instrument into position within the patient. The medical instrument can be guided inside a patient without continuously acquiring real time x-ray exposure.

Positions of the surgical instrument can be saved and pictorial representations of the surgical instrument in the saved positions can be superpositioned and displayed on the monitor display along with the pictorial representation of the surgical instrument in its current position. The surgeon can return the surgical instrument to a prior region of interest by realigning the pictorial representation of the current position with the pictorial representation of the saved position.

### BRIEF DESCRIPTION OF THE DRAWINGS

FIG. 1 illustrates a system for navigating and guiding a surgical instrument during a surgical procedure in accordance with an embodiment of the present invention.

FIG. 2 illustrates a surgical instrument for the system of FIG. 1 in accordance with an embodiment of the present invention.

FIG. 3 illustrates a surgical instrument for the system of FIG. 1 in accordance with an embodiment of the present invention.

FIG. 4 illustrates an image on a display with a saved position of a needle and stylet and a current position of the needle and stylet displayed in relation to a patient's anatomy.

FIG. 5 is a block diagram illustrating a method for navigating and guiding an access needle in accordance with an embodiment of the present invention.

### DETAILED DESCRIPTION OF THE INVENTION

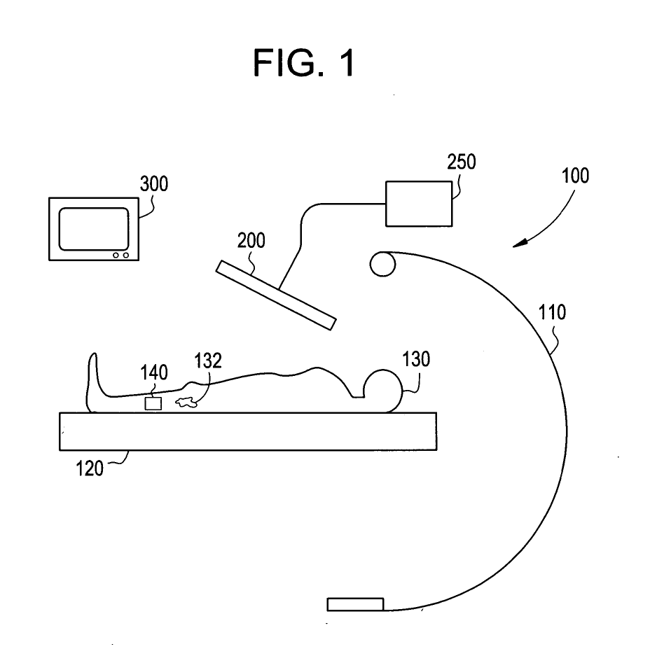

FIG. 1 illustrates a system 100 for navigating a surgical instrument during a procedure in accordance with an embodiment of the present invention. The system 100 includes an imaging device 110, a patient table 120, a patient 130, a region of interest 132, a transmitter 140, a surgical instrument 200, a monitor display 300, and a processing unit 250. The patient 130 is positioned on the surgical table 120 near an imaging device 120. The transmitter 140 is attached to the patient 130. The display 300 is positioned near the surgical table 120.

In operation, the imaging device 110 obtains x-ray images of the patient's bony anatomy. The x-ray images are transmitted to the processing unit 250. The transmitter 140 is attached to a portion of the patient's anatomy and transmits a signal. The signal is received by the surgical instrument 200. The surgical instrument 200 transmits data to the processing unit 250 that corresponds to the signal received from the transmitter 140. The processing unit 250 uses the data received from the surgical instrument 200 to determine the location of the surgical instrument 200 in relation to the transmitter 140. The processing unit 250 creates a pictorial representation of at least a portion of the surgical instrument 200 and combines the pictorial representation with the x-ray images of the patient's bony anatomy. The combined x-ray images are transmitted to the display 300. In the combined x-ray images, the position of the surgical instrument 200 is shown in relation to the patient's anatomy.

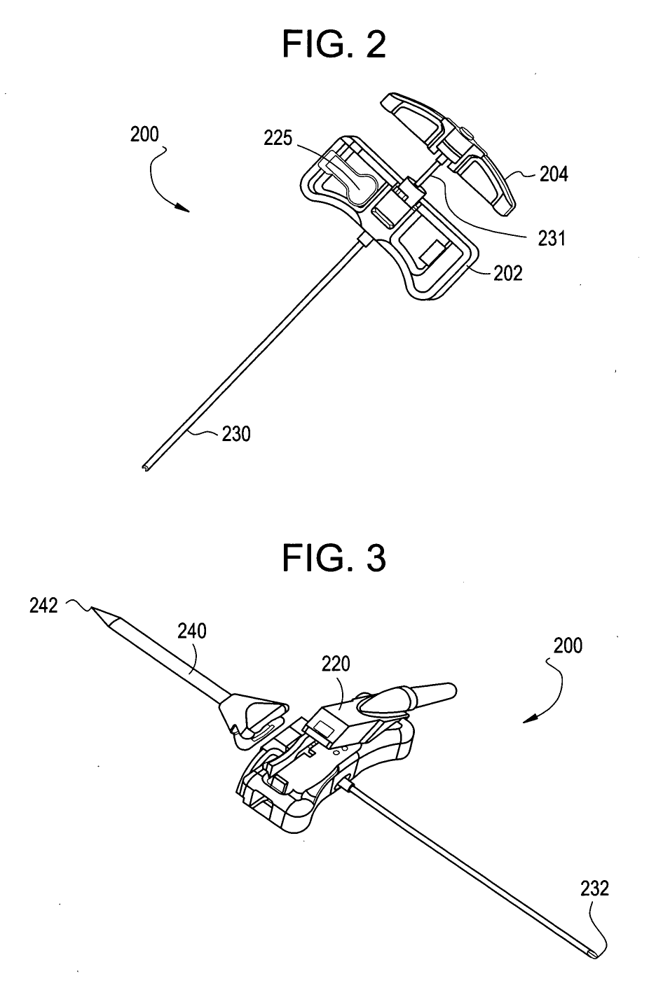

FIGS. 2 and 3 illustrate the surgical instrument 200 of FIG. 1 in accordance with an embodiment of the present invention. The surgical instrument 200 includes a lower assembly 202, a top assembly 204, a receiver 220, a save virtual tool image button 225, a needle 230, a stylet 231, a stylet tip 232, a back calibration insert post 240, and a calibration insert tip 242. The stylet 231 with the stylet tip 232 is connected to the top assembly 204. The needle 230 is connected to the lower assembly 202. The stylet 231 slides in the needle 230 and the stylet tip 232 projects out a hole in the end of the needle 230. The stylet 231 is slid within the needle 230 until the lower assembly 202 prevents the top assembly 204 from sliding further. The top assembly 204 and the lower assembly 202, in conjunction, form a handle for manipulating and positioning the surgical instrument 200. The receiver 220 is attached to the lower assembly 202. The save virtual tool image button 225 is located on the lower assembly 202. The back calibration insert post 240 is removably attached to the top assembly 204. The back calibration insert post 240 has a calibration insert tip 242 at a distal end.

In operation, the stylet 231 of the top assembly 204 is slid into the needle 230 until the top assembly 204 is prevented from sliding further by the lower assembly 202. A snap connector or other type of fastener is provided to secure the top assembly 204 in place when it is brought into contact with the lower assembly 202. After the top assembly 204 is attached to the lower assembly 202, the stylet tip 232 projects from an opening in the end of the needle 230.

The receiver 220 is attached to the lower assembly 202. To secure the receiver 220 to the lower assembly 202, a snap connector or other type of fastener is used. The receiver 220 can then be brought into contact with the lower assembly 202 and conveniently snapped into place on the lower assembly 202.

After the receiver 220 is attached to the lower assembly 202, the surgical instrument is calibrated to locate distal portions of the surgical instrument in relation to the receiver, the transmitter, and the patient's anatomy. To calibrate the surgical instrument, the stylet tip 232 is brought into contact with the transmitter 140. With the stylet tip 232 in contact with the transmitter 140, the receiver 220 transmits data to the processing unit 250. Because the stylet tip 232 is in the same location as the transmitter 140 and the processing unit 250 can determine the location of the receiver relative to the transmitter 140, the processing unit 250 is able to calculate the position of the stylet tip 232 and transmitter 140 relative to the receiver 220. The location of the stylet tip 232 relative to the receiver 220 is saved in the processing unit 250 for use during navigation and visualization of the surgical instrument during the surgical procedure.

A back calibration insert 240 is attached to the top assembly. The back calibration insert tip 242 of the back calibration insert post 240 is brought into contact with the transmitter 140. With the back calibration insert tip 242 in contact with the transmitter 140, the receiver 220 transmits data to the processing unit 250. Because the back calibration insert tip 242 is in the same location as the transmitter 140 and the dimensions of the back calibration insert 240 are known, the processing unit 250 can calculate the position of the back tip of the stylet 231 that is located where the back calibration insert post 240 is attached to the top handle assembly.

After the surgical instrument has been tip calibrated, the location of the stylet tip 232 can be tracked as the surgical instrument is moved. As the surgical instrument is moved, the receiver 220 receives signals from the transmitter 140. The receiver transmits data to the processing unit that represents the signals received from the transmitter. The processing unit 250 uses the data from the receiver to calculate the position of the receiver relative to the transmitter and the patient's anatomy. Because the position of the stylet tip relative to the receiver is known from the calibration procedure, the processing unit 250 can determine the location of the stylet tip 232 relative to the transmitter 140. Also, the processing unit 250 can determine the location of the transmitter 140 relative to patient anatomy and, thus, can use this to determine the position and orientation of portions of the surgical instrument relative to patient anatomy.

As presented earlier with regard to FIG. 1, the processing unit 250 then creates a pictorial representation of at least a portion of the surgical instrument 200 and combines the pictorial representation with the x-ray images of the patient's bony anatomy into display images. The combined display images are transmitted to the monitor display 300. In the display images, the position of the surgical instrument 200 can be shown in relation to the patient's anatomy.

For example, a system for navigating and guiding a surgical instrument such as that shown in FIGS. 1-3 can be used during various orthopedic and interventional procedures such as bone biopsies, guide wire placement, injections and aspirations. During such procedures, x-ray images of a patient's anatomy can be obtained using a C-arm imaging device. If a surgeon wants to obtain a biopsy from a vertebra in the spine of a patient, a transmitter can be attached near or on the patient's spine. To perform a biopsy, a needle with a sharp tip is directed to the vertebrae and a drill channel is created into the vertebrae. Rather than guide the needle while viewing the position of the stylet tip in real time x-ray images and subject the surgeon and patient to excessive doses of x-rays, a receiver can be attached to the handle of the needle and the needle/stylet can be guided by electromagnetic tracking with signals received from the transmitter.

Before guiding the needle/stylet combo with the transmitter and receiver, the system is calibrated. The tip of the needle/stylet combo is brought into contact with the transmitter that is attached to the patient's body. The processing unit determines the length of the needle/stylet combo and the location of stylet tip. After the system has been calibrated and verified, the needle and stylet can be inserted into a patient. A processing unit tracks movement of the receiver and needle. The processing unit adds a pictorial representation of the needle and stylet to the previously obtained x-ray images of the patient anatomy to produce combined images that illustrate the location and orientation of the needle and stylet relative to the patient's anatomy. The processing unit transmits the combined images to a monitor display that can be viewed by the surgeon during a procedure. By viewing the calculated position of the needle and stylet relative to the patient's anatomy as shown on the monitor display, a surgeon can navigate the tip of the stylet and needle to a desired target point in the patient's anatomy without having to produce another set of x-ray images.

When the desired target point has been reached, the needle and stylet can be used to drill a hole in a vertebrae or other bone. The top assembly 204 can be unlocked or unsnapped from the lower assembly 202 and the stylet can be removed from the needle. The lumen of the needle can then be used as a guideway channel to perform a biopsy, introduce a guide wire into a portion of the patient's anatomy, conduct an aspiration, inject materials such as steroids and bone cement, and perform various other procedures that utilize a guideway into a patient's anatomy.

Consequently, a surgeon can reduce exposure to x-ray radiation by navigating and guiding portions of the surgical instrument using a system with a transmitter and receiver to electromagnetically track movement of the surgical instrument rather than produce multiple x-ray images that show the position of the medical instrument as it is guided into place.

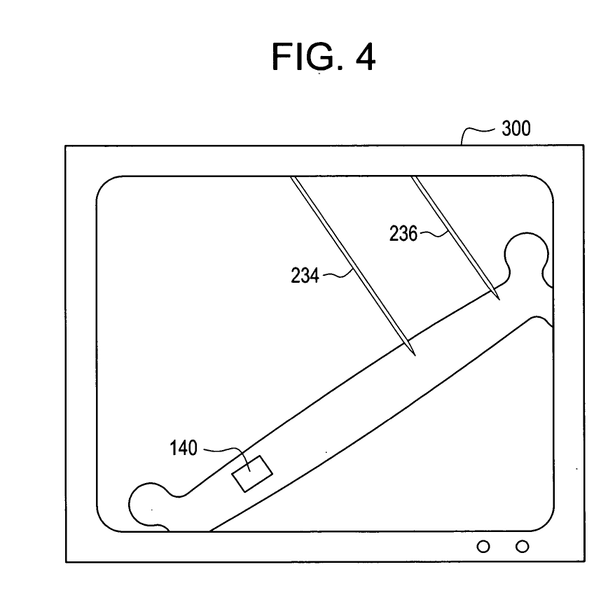

FIG. 4 illustrates an image on a display 300 with a saved position of the surgical instrument and a current position of the surgical instrument displayed. The image includes a transmitter 140, a save position 234, and a current position 236. The save virtual image button can be activated when a user desires to leave a virtual line indicating the last location of the surgical instrument in relation to the patient's anatomy. The location of the surgical instrument at the moment the button is pressed is then saved in the processing unit 250 and a pictorial representation of the surgical instrument at the saved location is displayed in the combined x-ray image on the monitor display. On the same x-ray image, the current real-time position 236 of the surgical instrument is displayed as it is moved around a region of interest. Displaying the save position 234 on the monitor display allows the user to conveniently return the surgical instrument to the previously saved position by subsequently aligning and overlapping the current position 236 displayed on the monitor display with the previously saved position 234. Thus, if a surgeon locates a point of interest in the patient's anatomy and desires to return the surgical instrument to that point of interest to perform a portion of the procedure at a later time, the surgeon can align the current position 236 of the surgical instrument with the previously saved position 234 shown on the monitor display and be assured that the surgical instrument has been returned to the original last point of interest.

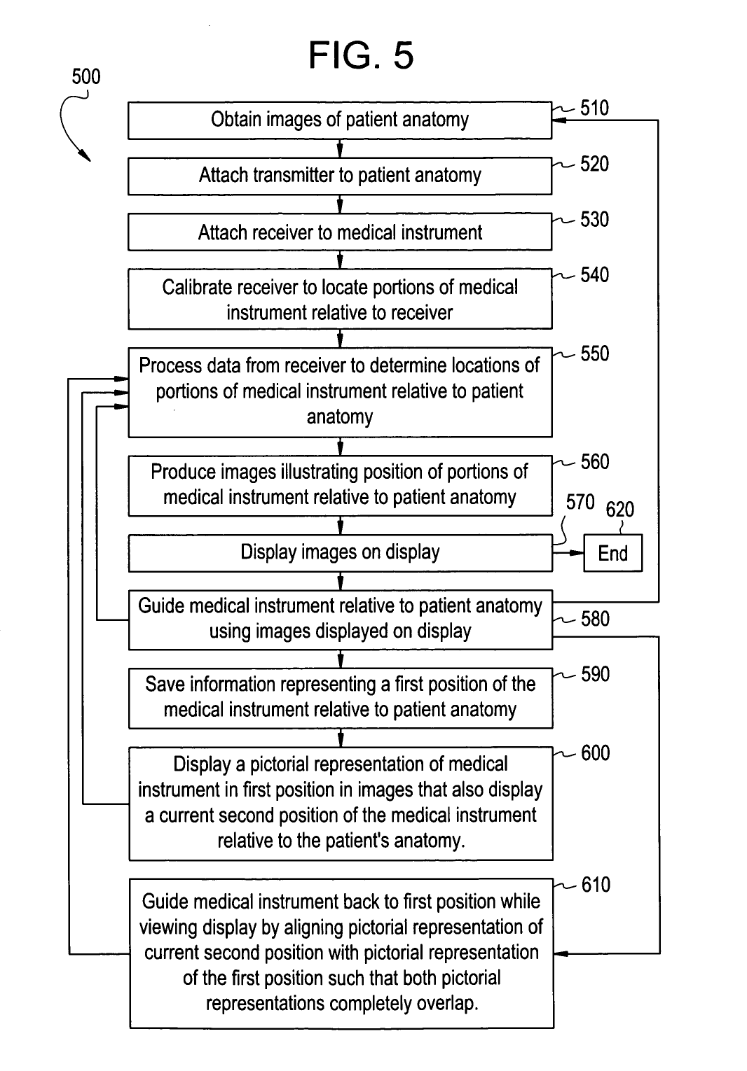

FIG. 5 is a flow chart illustrating a method 500 for navigating and guiding a surgical instrument during a surgical procedure in accordance with an embodiment of the present invention. In step 510, images are obtained of a patient's anatomy. The images may be obtained using procedures such as x-ray, MRI, ultrasound, or a combination of them all.

In step 520, a transmitter is attached to a portion of the patient's anatomy. The transmitter emits signals to be received by a receiver. More than one transmitter may be attached to a patient's anatomy that emit signals for receipt by a single receiver or multiple receivers.

In step 530, a receiver is attached to a surgical instrument. The receiver receives signals from the transmitter and transmits data to a processing unit. The receiver may be attached to the surgical instrument with various types of receptacles or fasteners. The receiver may be removably attached to the surgical instrument with a snap connector or a latching fastener. In the alternative, the receiver can be built into the surgical instrument and supplied as a part of the surgical instrument.

In step 540, the receiver is tip calibrated. The receiver is calibrated to verify its position relative to the transmitter. The receiver is also calibrated to determine the location of portions of the surgical instrument relative to the receiver and transmitter. For example, the needle and stylet combination of FIGS. 2 and 3 would be calibrated to determine the location of the tip of the needle/stylet combination.

In step 550, data transmitted from the receiver is processed by a processing unit. As the receiver is moved, the receiver transmits data to the processing unit regarding signals received from the transmitter. The processing unit uses the data from the receiver to determine the location of the surgical instrument relative to the patient's anatomy.

In step 560, the processing unit produces images that illustrate the position of the medical instrument relative to the patient's anatomy. The processing unit produces images with a pictorial representation of the surgical instrument and the images of the patient's anatomy. The images produced by the processing unit represent a real time approximation of the position of the surgical instrument in relation to the patient's anatomy. For example, the position of the access needle of FIGS. 2 and 3 would be shown relative to the patient's anatomy.

In step 570, the images produced by the processing unit are transmitted to a display device. The images are displayed on the display device. In step 580, a surgeon views the images during a surgical procedure and guides the surgical instrument toward a region of interest by watching the pictorial representation of the surgical instrument move in relation to patient anatomy in the images. While the surgical instrument is being moved, steps 550-570 are continuously repeated to update the image on the monitor display. If the surgical instrument is in position, then the method proceeds to step 620 and ends. For example, a surgeon would guide the access needle of FIGS. 2 and 3 by watching the pictorial representation of the access needle in relation to the patient's anatomy as shown on a display.

In step 590, information representing a first position of the medical instrument relative to a patient's anatomy is saved. To save the information, a button in the handle is pressed and position information is saved in the processing unit. For example, if a surgeon locates a region of interest in a patient's anatomy and believes it has potential for treatment or uses as a guide for surgical tools, the surgeon can save the position information for the access needle of FIGS. 2 and 3. The surgeon can then proceed to insert the access needle at other regions of interest and see if a location for treatment that is better than the first position exists.

In step 600, the processing unit adds a pictorial representation of the surgical instrument in the saved first position to the images displayed on the monitor display. With the first position displayed, the images on the display show the patient's anatomy, a pictorial representation of the surgical instrument in the first position, and a pictorial representation of the surgical instrument in its current position or a second position.

In step 610, the surgical instrument is guided back to the first position for which the location information was saved. While guiding the surgical instrument in step 580, a surgeon may desire to return the surgical instrument to a previous location or region of interest. If the location information was saved as in step 590, then the method can proceed to step 610. While watching the display, the surgeon can move the surgical instrument until the pictorial representation of the surgical instrument in the second position (current position) aligns with and overlaps the pictorial representation of the surgical instrument in the second position (saved position). Once the pictorial representations overlap, the surgical instrument has been returned to the location of the first position. Thus, a surgeon using the access needle of FIGS. 2 and 3 can conveniently return the access needle to the saved position knowing the access needle has been returned to the saved location and orientation.

In an alternative embodiment, a device other than an access needle may be used as the surgical instrument. For example, a scalpel may be used as the surgical instrument. A receiver would be attached to the scalpel. The scalpel would be calibrated by touching the tip of the scalpel to the transmitter. Movement of the scalpel would be tracked and displayed on a monitor display along with x-ray images of the patient's anatomy. A surgeon could then guide the scalpel around a patient's anatomy by using the displayed image to determine the position of the scalpel relative to portions of a patient's anatomy.

Consequently, using a transmitter and receiver allows medical personnel to guide a needle and stylet with a minimally invasive procedure while minimizing exposure to x-ray radiation. Also, using a system that passes electromagnetic signals between a transmitter and receiver minimizes dependence upon maintaining unobstructed line-of-sight paths between a surgeon's eyes and the region of interest or between the transmitter and receiver.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

What is claimed is:

1. A system for guiding an access needle during a surgical procedure including:
an x-ray imaging device that produces x-ray images of a patient's anatomy;
a surgical instrument with a handle, an access needle and a stylet;
a transmitter positioned proximal to the patient's anatomy;
a receiver positioned in said handle of said surgical instrument;
a processing unit that receives said x-ray images from said x-ray imaging device and data from said receiver, wherein said processing unit produces display images that show the position of said surgical instrument in relation to at least a portion of the patient's anatomy; and
a display that receives said display images from said processing unit and displays the images.

2. The system of claim 1 wherein said handle of said surgical instrument includes a top assembly attached to said stylet and a lower assembly attached to said access needle, and the stylet slides within the access needle and protrudes from an opening in the distal end of said access needle.

3. The system of claim 2 wherein said receiver is removably attached to said lower assembly.

4. The system of claim 2 wherein said top assembly interlocks with said lower assembly.

5. The system of claim 1 including a button positioned on said handle that is pressed to save data associated with the location of the surgical instrument.

6. The system of claim 5 wherein said processing unit stores the data associated with the location of the surgical instrument at the time the button is pressed and produces display images with a pictorial representation of a portion of the surgical instrument at the location where the surgical instrument was positioned at the time the save button was pressed along with a pictorial representation of the surgical instrument at its most recent detected location.

7. The system of claim 1 wherein said transmitter emits electromagnetic signals, said receiver receives said electromagnetic signals and transmits data including position information to said processing unit, and said processing unit determines the location of said receiver with respect to said transmitter.

8. The system of claim 1 further including a calibration insert attached to said handle and used to calibrate the location of the receiver with respect to the transmitter.

9. The system of claim 8 wherein said calibration insert is removably attached to said handle.

10. The system of claim 1 wherein said transmitter is attached to a portion of the patient's anatomy.

11. A method for guiding an access needle during a surgical procedure including:
obtaining x-ray images of a patient's anatomy;
positioning a transmitter proximal to the patient's anatomy;
providing a medical instrument with a handle, an access needle and a stylet;
attaching a receiver to said handle;
processing data from said receiver with a processing unit that receives said x-ray images from said x-ray imaging device and data from said receiver, wherein said processing unit produces images that show said medical instrument in relation to at least a portion of the patient's anatomy shown in said x-ray images; and
displaying said images from said processing unit on a display.

12. The method of claim 11 wherein said handle of said surgical instrument includes a top assembly attached to said stylet and a lower assembly attached to said access needle.

13. The method of claim 12 wherein said attaching step further includes snapping said receiver to said handle with a snap connector.

14. The method of claim 12 further including interlocking said top assembly with said lower assembly.

15. The method of claim 11 further including pressing a button to save data associated with the location of the surgical instrument.

16. The method of claim 15 further including storing said data associated with the location of the surgical instrument at the time the button is pressed and producing display images with a pictorial representation of a portion of the surgical instrument at the location where the medical instrument was positioned at the time the save button was pressed along with a pictorial representation of the surgical instrument at its most recent detected location.

17. The method of claim 15 wherein said displaying step includes displaying an image with a saved position, a current position, and the patient's anatomy.

17. The method of claim 11 further including guiding a portion of said surgical instrument to a region of interest in the patient's anatomy.

18. The method of claim 11 further including calibrating the location of portions of the surgical instrument with respect to the transmitter.

19. The method of claim 18 wherein said calibrating step includes attaching a calibration insert to said surgical instrument.

20. The method of claim 11 wherein said positioning step further includes attaching said transmitter to a portions of the patient's anatomy.
